(19) United States
(12) Patent Application Publication
(10) Pub. No.: US 2007/0149919 A1
(43) Pub. Date: Jun. 28, 2007

(54) Concentric thin wall surgical irrigation tubing

(75) Inventors: James T. Perkins, St. Charles, MO (US); Michael Versemann, St. Charles, MO (US); Jeffery A. Knight, Maryland Heights, MO (US)

Correspondence Address: Michael L. Smith;Bausch & Lomb Incorporated, One Bausch & Lomb Place, Rochester, NY, 14604, US

(73) Assignee: Bausch & Lomb Incorporated

(21) Appl. No.: 11/708,247

(22) Filed: Feb. 20, 2007

Related U.S. Application Data
(63) Continuation-in-part of application No. 11/186,573, filed on Jul. 21, 2005.

(51) Int. Cl. A61M 3/00

(52) U.S. Cl. 604/43

(57) ABSTRACT

A length of ophthalmic surgical irrigation tubing 14 includes an inner lumen 16 for transporting fluid from a source 24 to a surgical handpiece 26. The inner lumen 16 is formed by an inner shell 18. A flexible outer shell 20 spans and surrounds a length of the inner shell 18. A plurality of flexible webs 22 is connected between the inner shell 18 and the outer shell 20. The combination of the outer shell and the webs provide support to the inner shell to resist kinking of the inner shell during surgery.

### CROSS-REFERENCE TO RELATED APPLICATIONS

This application is a continuation-in-part of and claims priority to U.S. patent application Ser. No. 11/186,573, filed Jul. 21, 2005, the entire disclosure of which is incorporated herein by reference.

### BACKGROUND OF THE INVENTION

1. Field of the Invention

The present invention is directed towards surgical tubing. More specifically, the present invention is directed towards surgical tubing for use in ophthalmic surgery for transporting irrigation fluid to a surgical site.

2. Description of Related Art

In surgery, particularly in ophthalmic surgery, the use of irrigation tubing for delivering fluid from an irrigation source to a surgical handpiece and eventually a surgical site is commonly required. In ophthalmic surgery, irrigation fluid is necessary for several reasons, including keeping the eye inflated and preventing collapse of the eye, which can cause serious damage.

It is desirable to have flexible tubing that can expand as fluid is introduced into the tubing, such that the tubing can act as an accumulator during occlusion of such instruments as a phacoemulsification handpiece. In this way, a sufficient supply of irrigating fluid is available quickly to a surgical site upon removal of the occlusion (post occlusion surge) to assist in preventing collapse of the eye. However, one of the significant downsides to very flexible or thin walled tubing is that such tubing is easily kinked which can prevent the flow of fluid into the eye, which is an unacceptable risk.

In order to prevent or at least minimize the chance of the tubing kinking and cutting off the supply of fluid, the thickness of the irrigation tubing is often built-up, such that the tubing is rigid or stiff enough to not easily kink. However, the build-up to make the tubing sufficiently stiff to prevent kinking, then may cause an undesirable torque effect on the handpiece that is being used by the surgeon. The weight and the stiffness of the tubing tends to pull on an end of the handpiece, which can fatigue a surgeon's hand as well as continually bias the handpiece in a direction that may not be desired by the surgeon. This may result in the surgeon being uncomfortable during delicate surgery, such as routinely incurred in ophthalmic surgery. Therefore, it would be desirable to have a thin wall irrigation tubing for ophthalmic surgery to act as a fluid accumulator so that sufficient fluid can be supplied to the handpiece to prevent collapse of the eye, but yet still be resistant to bending and kinking, to ensure a constant flow of irrigation fluid into the surgical handpiece.

### BRIEF DESCRIPTION OF THE DRAWING

FIG. 1 is a prior art depiction of thin wall surgical tubing being kinked to prevent the flow of irrigation fluid;

FIG. 2 is a cross-sectional view of irrigation tubing in accordance with the present invention;

FIG. 3 is a partial perspective view of irrigation tubing in accordance with the present invention;

FIG. 4 is a partial elevation view of surgical tubing in accordance with the present invention being bent; and

FIG. 5 is a schematic diagram of a surgical system using tubing in accordance with the present invention.

### DETAILED DESCRIPTION OF THE PREFERRED EMBODIMENT

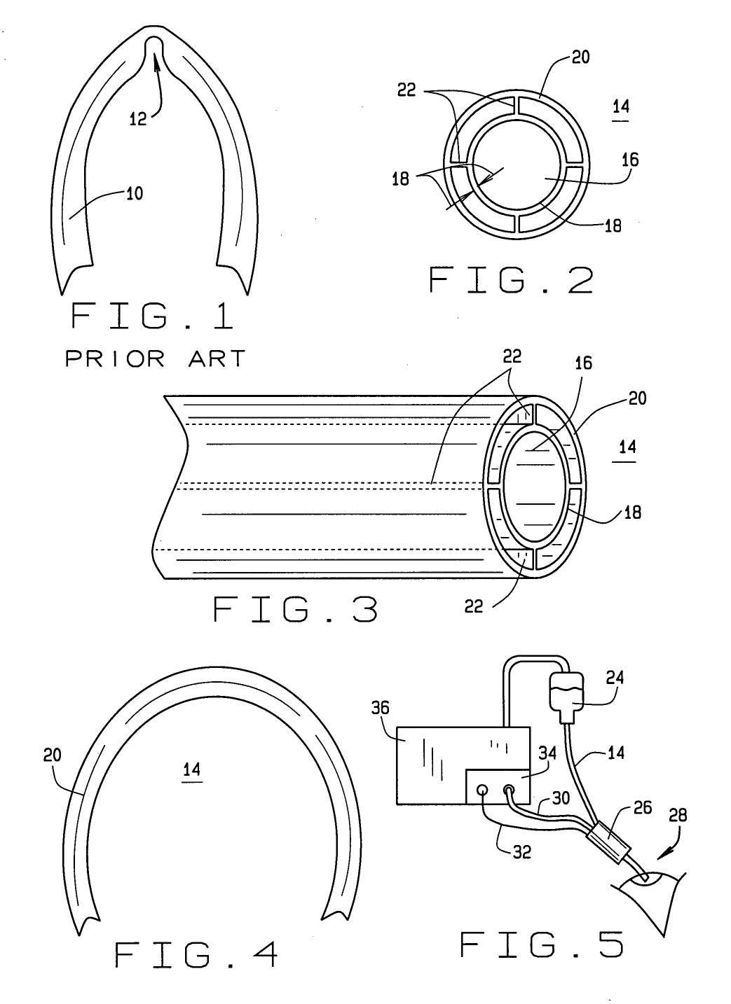

FIG. 1 shows prior art tubing 10 being bent with a kink, shown generally at 12, which causes a flow of fluid through tubing 10 to be cut-off at 12. As discussed above, this is highly undesirable and can lead to significant injury to an eye during surgery (corneal burn). Yet having thin wall tubing is desirable, in that the tubing can then act as a fluid accumulator during surgery. This accumulator effect ensures that a sufficient supply of fluid is available to a surgical handpiece upon the removal of an occlusion, to help prevent collapse of the eye post-occlusion. Historically, there has been a trade-off made between the flexibility of the tubing and the amount of rigidity required to prevent kinking, such that the tubing is flexible enough not to unacceptably torque the handpiece being used by the surgeon but yet be rigid enough not to easily kink.

The present invention, a cross-section of which is shown at FIG. 2, includes a length of surgical irrigation tubing 14, as shown in cross-section. The tubing includes an inner lumen 16 for transporting fluid from a source to a surgical handpiece. The inner lumen is formed by an inner shell 18 and has a desired thickness shown at arrows 18 less than 0.015 inches. The inner shell 18 preferably is thin enough so that shell 18 is compliant and acts as an accumulator to have a sufficient supply of irrigation fluid to deliver to a handpiece during surgery.

The tubing 14 has a flexible outer shell 20 spanning and surrounding a length of the inner shell 18. Outer shell 20 is preferably formed simultaneously with inner shell 18 via a known extrusion process of silicone, PVC, polyurethane, or other suitable material. The flexible outer shell 20 is preferably of the same thickness as inner shell 18 but could be thicker or thinner depending on the amount of flexibility desired in tubing 14.

A plurality of flexible webs 22 is connected between the inner shell 18 and the outer shell 20. The outer shell 20 in combination with the webs 22 provides support to the inner shell 18 to resist kinking of the inner shell 18 during surgery. Obviously, more or fewer than the four webs 22 shown may be used. The more webs used, the more rigid or resistant to bending the tubing 14 will be. This combination of an outer shell and webs connected to a compliant inner shell allows the tubing 14 to be flexible but to resist kinking and therefore ensure a continuous supply of irrigating fluid to a surgical site.

FIG. 3 shows a partial perspective view of the tubing 14 showing the webs 22 connected between the outer shell 20 and the inner shell 18. While the webs 22 are shown running along the longitudinal axis of the tubing 14, webs may also run in spirals along tubing 14. While the outer shell 20 and webs 22 have been shown to span the entire length of tubing 14, it is to be understood that they may not span the entire length of tubing 14, but rather only a substantial length in order to provide sufficient resistance to kinking of the tubing during surgery. Tubing 14 also provides the benefit of being able to receive a standard barb fitting on the end of the tubing for connection to a fluid source or a surgical handpiece.

FIG. 4 shows an elevation view of a partial length of tubing 14 being bent that resists bending and therefore prevents or minimizes kinking of the tubing 14 during use in surgery, unlike the tubing of FIG. 1. It is desirable that the thickness of the inner shell 18 be a maximum of 0.015 inches. This is compared to a standard prior art irrigation tube thickness of 0.021 inches. By making the thickness of the tubing thin, tubing 14 and specifically inner shell 18, can act as a fluid accumulator to provide a sufficient supply of fluid quickly to the handpiece upon the removal of an occlusion from the handpiece, such as a phacoemulsification handpiece during surgery. This is quite important, in order to prevent collapse of the eye, which can cause significant and perhaps irreversible damage to the eye.

FIG. 5 shows a length of flexible irrigation tubing 14 having an inner lumen 16 for transporting fluid 24 to a surgical handpiece 26. Surgical handpiece 26 is connected to the tubing 14 for performing an ophthalmic surgical procedure, including delivering the fluid to a surgical site, shown generally at 28.

Surgical handpiece 26, is also connected to tubing 30, and power cord 32, which in turn are connected to surgical pump 34, which forms a part of a surgical system 36.

We claim:

1. A length of ophthalmic surgical irrigation tubing comprising:
a length of flexible irrigation tubing having an inner lumen for transporting fluid from a source to a surgical handpiece;
wherein the inner lumen is formed by an inner shell;
a flexible outer shell spanning and surrounding a length of the inner shell;
a plurality of flexible webs connected between the inner shell and the outer shell; and
wherein the outer shell in combination with the webs provides support to the inner shell to resist kinking of the inner shell during surgery.

2. The invention of claim 1, wherein the irrigation tubing is formed by extrusion.

3. The invention of claim 2, wherein the irrigation tubing is formed of one of silicone, PVC, or polyurethane.

4. The invention of claim 1, wherein the inner shell has a thickness of less than 0.015 inches.

5. An ophthalmic surgical system comprising:
a length of flexible irrigation tubing having an inner lumen for transporting fluid from a source to a surgical handpiece;
the surgical handpiece connected to the tubing for performing an ophthalmic surgical procedure, including delivering the fluid to a surgical site; and
wherein the tubing includes an inner shell forming the inner lumen and an outer shell spanning and surrounding a length of the inner shell and a plurality of webs connected between the inner shell and the outer shell, such that the outer shell in combination with the webs provides support to the inner shell to resist kinking of the inner shell during surgery.

6. The invention of claim 5, wherein the irrigation tubing is formed by extrusion.

7. The invention of claim 5, wherein the irrigation tubing is formed of one of silicone, PVC, or polyurethane.

8. The invention of claim 5, wherein the inner shell has a thickness of less than 0.015 inches.

9. The invention of claim 5, wherein the surgical handpiece includes one or more of a phacoemulsification handpiece, an irrigation and aspiration handpiece, and an irrigation handpiece.
